(19) United States
(12) Patent Application Publication
(10) Pub. No.: US 2025/0033189 A1
(43) Pub. Date: Jan. 30, 2025

(54) INFORMATION PRESENTATION SYSTEM, INFORMATION PRESENTATION DEVICE, INFORMATION PRESENTATION METHOD, AND PROGRAM

(71) Applicants: KEIO UNIVERSITY, Minato-ku, Tokyo (JP); MOTION LIB, INC., Kawasaki-shi, Kanagawa (JP); TERUMO KABUSHIKI KAISHA, Shibuya-ku, Tokyo (JP)

(72) Inventors: Kouhei OHNISHI, Kawasaki-shi, Kanagawa (JP); Takahiro MIZOGUCHI, Kawasaki-shi, Kanagawa (JP); Shin MAKI, Ashigarakami-gun, Kanagawa (JP); Yoshiyuki HABU, Ashigarakami-gun, Kanagawa (JP); Toshihiro FUJII, Ashigarakami-gun, Kanagawa (JP)

(73) Assignees: KEIO UNIVERSITY, Minato-ku, Tokyo (JP); MOTION LIB, INC., Kawasaki-shi, Kanagawa (JP); TERUMO KABUSHIKI KAISHA, Shibuya-ku, Tokyo (JP)

(21) Appl. No.: 18/705,056

(22) PCT Filed: Oct. 10, 2022
(86) PCT No.: PCT/JP2022/037746

§ 371 Date: Apr. 26, 2024

(30) Foreign Application Priority Data
Oct. 29, 2021 (JP) 2021-178388

(51) Int. Cl. B25J 3/00; G06F 3/01

(52) U.S. Cl. CPC B25J 3/00; G06F 3/016

(57) ABSTRACT

An object of the present invention is to provide further assistance in addition to assistance by tactile force transmission. An information presentation system includes a master device to which an operation by an operator is input and a slave device that operates in accordance with the operation input to the master device. Furthermore, the information presentation system includes a tactile force transmission unit, a calculation unit, and a presentation unit. The tactile force transmission unit controls tactile force transmission in the master device and the slave device. The calculation unit calculates physical characteristics of a substance in contact with the slave device on the basis of an external force input from the environment to the slave device while the slave device maintains a predetermined motion state. The presentation unit presents the physical characteristics of the substance calculated by the calculation unit.

### TECHNICAL FIELD

The present invention relates to an information presentation system, an information presentation device, an information presentation method, and a program.

### BACKGROUND ART

Conventionally, there is known a technique of bilateral control by which, in a master device to which an operation of an operator is input and a slave device that operates in accordance with an operation input to the master device, a reaction force corresponding to an operation of the slave device is transmitted to the master device as a tactile force. A technique related to such bilateral control is disclosed in, for example, Patent Literature 1.

### CITATION LIST

### Patent Literature

Patent Literature 1: JP S64-34686 A

### SUMMARY OF INVENTION

### Technical Problem

According to the general technique as described above, it is possible to assist an operator's operation by transmitting tactile force between the master device and the slave device.

However, it is desirable that further assistance can be provided in addition to such assistance by tactile force transmission.

The present invention has been made in view of the aforementioned circumstances. An object of the present invention is to provide further assistance in addition to assistance by tactile force transmission.

### Solution to Problem

In order to solve the aforementioned problems, an information presentation system according to one aspect of the present invention including
a master device to which an operation by an operator is input and a slave device that operates in accordance with the operation input to the master device, includes:
a control means that controls tactile force transmission in the master device and the slave device;
a calculation means that calculates physical characteristics of a substance in contact with the slave device based on an external force input from an environment to the slave device while the slave device maintains a predetermined motion state; and
a presentation means that presents the physical characteristics of the substance calculated by the calculation means.

### Advantageous Effects of Invention

According to the present invention, it is possible to provide further assistance in addition to assistance by tactile force transmission.

### BRIEF DESCRIPTION OF DRAWINGS

FIG. 1 is a schematic diagram illustrating an overall configuration of an information presentation system 1 according to an embodiment of the present invention.

FIG. 2 is a schematic diagram illustrating a basic principle of tactile force transmission control executed by an information presentation device 30.

FIG. 3 is a block diagram illustrating a hardware configuration of a control system in the information presentation system 1.

FIG. 4 is a schematic diagram illustrating a hardware configuration of an information processing device constituting the information presentation device 30.

FIG. 5 is a block diagram illustrating the functional configuration of the information presentation system 1.

FIG. 6 is a flowchart for describing a flow of information presentation processing executed by the information presentation device 30.

FIG. 7 is a schematic diagram illustrating a time-series change in an external force input from the environment to a slave device 20 when the slave device 20 comes into contact with a substance.

FIG. 8 is a schematic diagram illustrating a configuration of the information presentation system 1 that performs information presentation processing after an operator manually inserts a catheter of the slave device 20.

### DESCRIPTION OF EMBODIMENTS

Hereinafter, an embodiment of the present invention will be described with reference to the drawings.

### [Configuration]

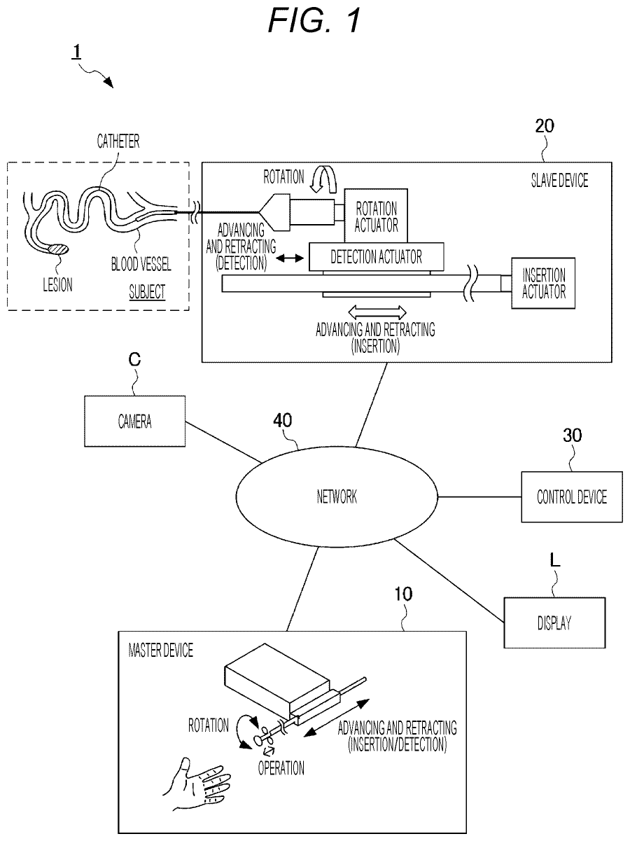

FIG. 1 is a schematic diagram illustrating an overall configuration of an information presentation system 1 according to an embodiment of the present invention.

As illustrated in FIG. 1, the information presentation system 1 according to the present embodiment is configured as a master-slave system including a master device 10 and a slave device 20 that are mechanically separated. As an example, in the information presentation system 1 in the present embodiment, it is assumed that the master device 10 constitutes a manipulator operated by the operator, and the slave device 20 constitutes a catheter system including an end effector to be inserted into a subject.

In FIG. 1, the information presentation system 1 includes the master device 10, the slave device 20, and the information presentation device 30, and the master device 10, the slave device 20, and the information presentation device 30 is configured to perform wired or wireless communication via a network 40. Note that the information presentation system 1 can appropriately include a display L and a plurality of cameras C. As the cameras C, various imaging devices such as a video camera that captures an external appearance of a subject into which the slave device 20 is inserted or an X-ray camera that captures the inside of the subject (for example, a blood vessel and an organ of the subject) by X-ray can be used. Furthermore, a plurality of displays L that displays various images captured by the plurality of cameras C and various types of information output from the information presentation device 30 can be provided.

The master device 10 receives an operation similar to an operation for a conventional catheter configured mechanically, and detects a position of a movable portion (a movable member of the manipulator, or the like) moved by the input operation. The master device 10 transmits information representing the detected position of the movable portion to the information presentation device 30. Furthermore, the master device 10 outputs reaction force by an actuator in response to the input operation in accordance with an instruction from the information presentation device 30.

Specifically, the master device 10 receives an operation of advancing and retracting the catheter (for example, an operation of inserting the catheter into a blood vessel, an operation of slightly moving the catheter to detect tactile force near a lesion, or the like), an operation of axially rotating the catheter (for example, an operation of changing a direction of the end effector, or the like), and an operation of operating the end effector (for example, in a case where the end effector is a balloon, an operation of expanding and contracting the balloon, and in a case where the end effector is a pair of forceps or the like, an operation of opening and closing the pair of forceps, or the like), and applies reaction force to these operations, and transmits, to the information presentation device 30, information representing a position of the movable portion moved by each operation.

The slave device 20 drives an actuator in accordance with an instruction from the information presentation device 30 to perform an operation corresponding to an operation input to the master device 10, and detects a position of a movable portion (a movable element of the actuator, the catheter moved by the actuator, or the like) moved by the operation. When the slave device 20 operates, various types of external force are input to the slave device 20 from an environment. As a result, the position of the movable portion in the slave device 20 indicates a result of the various types of external force acting on an output of the actuator. Then, the slave device 20 transmits information representing the detected position of the movable portion to the information presentation device 30. Here, the various external forces input to the slave device 20 from the environment include, for example, a resistance force in the thrust direction received from the blood vessel by the catheter inserted into the subject, and an abutment force when a guide wire, an end effector, or the like disposed at the distal end of the catheter comes into contact with a lesion, an organ, or a blood vessel.

The information presentation device 30 includes, for example, an information processing device such as a personal computer (PC) or a server computer, and controls the master device 10, the slave device 20, the display L, and the cameras C. For example, the information presentation device 30 executes control for acquiring positions of the movable portions of the master device 10 and the slave device 20 (rotation angles of the actuators detected by rotary encoders, advancing and retracting positions of the movable portions detected by linear encoders, or the like), and transmitting tactile force between the master device 10 and the slave device 20.

When operating the master device 10 and the slave device 20 as the master-slave system, the information presentation device 30 in the present embodiment performs coordinate transformation (transformation by a transformation matrix) of parameters (input vectors) of a real space calculated on the basis of information representing the positions of the movable portions (information representing positions of movable elements of the actuators, positions of members moved by the actuators, or the like) into a virtual space in which a position and force can be handled independently. That is, coordinate transformation of the input vectors from the real space of an oblique coordinate system in which a position and force are related to each other to the virtual space of an orthogonal coordinate system in which a position and force are independent from each other is performed. The parameters calculated by the coordinate transformation represent state values of positions and force corresponding to the input vectors in the virtual space. Then, in the virtual space after the coordinate transformation, the information presentation device 30 performs an arithmetic operation to cause the state values of the positions and the force calculated from the input vectors to follow the respective target values of the positions and the force for performing control of the positions and the force (here, tactile force transmission), and performs inverse transformation (transformation by an inverse matrix of the transformation matrix) to return an arithmetic operation result to the real space. Moreover, the information presentation device 30 implements the master-slave system that transmits tactile force between the master device 10 and the slave device 20 by driving each actuator on the basis of the parameters (a current command value and the like) of the real space acquired by the inverse transformation.

Note that, since the position and a velocity (or acceleration) or an angle and an angular velocity (or angular acceleration) are parameters that can be replaced by a differential and integral operation, it is possible to appropriately replace them with the velocity, the angular velocity, or the like when processing related to the position or the angle is performed.

In such a configuration, the information presentation system 1 according to the present embodiment implements the master-slave system that transmits tactile force between the master device 10 and the slave device 20 as described above, and performs the information presentation processing. Here, the information presentation processing is a series of processing of presenting to the slave device 20 the physical characteristics of the substance in contact with the slave device 20 on the basis of the external force input from the environment in a case where the predetermined action is executed by transmitting the tactile force.

Specifically, in the information presentation processing, the information presentation system 1 controls the tactile force transmission in the master device 10 and the slave device 20. In addition, the information presentation system 1 calculates physical characteristics of a substance in contact with the slave device 20 on the basis of an external force input from the environment to the slave device 20 while the slave device 20 maintains a predetermined motion state. Further, the information presentation system 1 presents the calculated physical characteristics of the substance.

As described above, the information presentation system 1 can present the physical characteristics of the substance in contact with the slave device 20 on the basis of an external force input from the environment to the slave device 20 in a quantitative state in which the slave device 20 maintains a predetermined motion state. Therefore, for example, the physical characteristics of a substance such as a blood vessel that cannot be directly touched by the operator can be quantitatively presented as information (that is, the tactile information) obtained by the slave device 20 in contact with the substance, whereby it is possible to further assist the operation of the operator. In addition to the operation by the operator, it is possible to further assist a person who performs various analyses, tests, and the like using the presented physical characteristics.

Therefore, according to the information presentation system 1, it is possible to solve the problem of providing further assistance in addition to assistance by tactile force transmission.

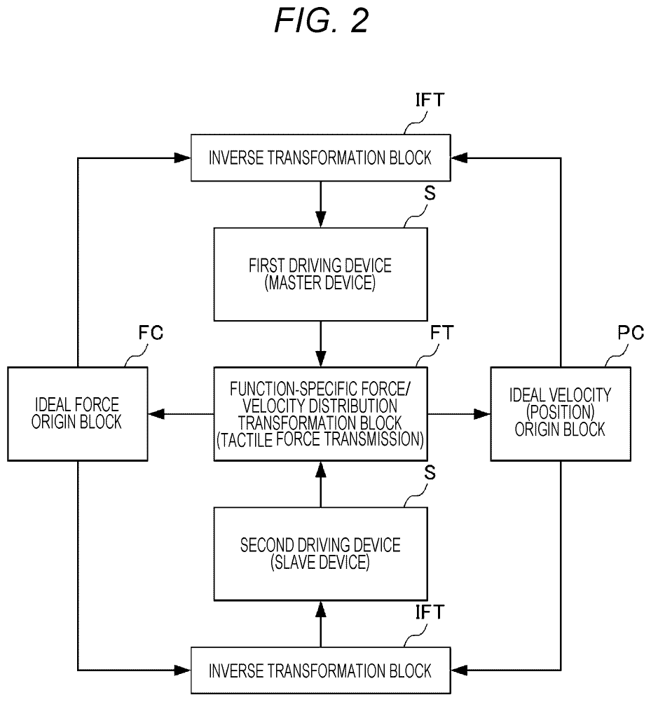

FIG. 2 is a schematic diagram illustrating a basic principle of tactile force transmission control executed by the information presentation device 30.

The basic principle illustrated in FIG. 2 is to determine an operation of the actuator by performing an arithmetic operation in at least one domain of a velocity or force with information representing a position of the movable portion (current position of the movable portion) as an input.

That is, the basic principle of the present invention is represented as control rules including a control object system S, a function-specific force/velocity distribution transformation block FT, at least one of an ideal force origin block FC or an ideal velocity origin block PC, and an inverse transformation block IFT.

The control object system S is the master device 10 or the slave device 20 including the actuator, and controls the actuator on the basis of the acceleration or the like. Here, as described above, since the acceleration, the velocity, and the position are physical quantities that can be mutually transformed by differentiation and integration, any of the acceleration, the velocity, and the position may be used for the control. Here, it is assumed that the control rules are expressed by mainly using the velocity calculated from the position.

The function-specific force/velocity distribution transformation block FT is a block that defines transformation of control energy in the domains of the velocity and the force, which is set in accordance with a function of the control object system S. Specifically, the function-specific force/velocity distribution transformation block FT defines coordinate transformation whose inputs are a value serving as a reference (reference value) for the function of the control object system S and a current position of the movable portion. The coordinate transformation is generally transformation of an input vector whose elements are a reference value and a current velocity to an output vector including a velocity for calculating a control target value of a velocity, and transformation of an input vector whose elements are a reference value and current force to an output vector including force for calculating a control target value of force. Specifically, the coordinate transformation in the function-specific force/velocity distribution transformation block FT can be generalized and represented as in the following Expressions (1) and (2).

### [Math. 1]

$$( \begin{matrix}{\dot{x}}_{1} \\ {\dot{x}}_{2} \\ ⋮ \\ {\dot{x}}_{n - 1} \\ {\dot{x}}_{n}\end{matrix} ) = ( \begin{matrix}{h}_{1 a} & {h}_{1 b} & … & {h}_{1 ( m - 1 )} & {h}_{1 m} \\ {h}_{2 a} & {h}_{2 b} & … & {h}_{2 ( m - 1 )} & {h}_{2 m} \\ ⋮ & ⋮ & ⋱ & ⋮ & ⋮ \\ {h}_{( n - 1 ) a} & {h}_{( n - 1 ) b} & … & {h}_{( n - 1 ) ( m - 1 )} & {h}_{( n - 1 ) m} \\ {h}_{na} & {h}_{nb} & … & {h}_{n ( m - 1 )} & {h}_{nm}\end{matrix} ) ( \begin{matrix}{\dot{x}}_{a} \\ {\dot{x}}_{b} \\ ⋮ \\ {\dot{x}}_{m - 1} \\ {\dot{x}}_{m}\end{matrix} ) \qquad ( 1 )$$

$$( \begin{matrix}{f}_{1} \\ {f}_{2} \\ ⋮ \\ {f}_{n - 1} \\ {f}_{n}\end{matrix} ) = ( \begin{matrix}{h}_{1 a} & {h}_{1 b} & … & {h}_{1 ( m - 1 )} & {h}_{1 m} \\ {h}_{2 a} & {h}_{2 b} & … & {h}_{2 ( m - 1 )} & {h}_{2 m} \\ ⋮ & ⋮ & ⋱ & ⋮ & ⋮ \\ {h}_{( n - 1 ) a} & {h}_{( n - 1 ) b} & … & {h}_{( n - 1 ) ( m - 1 )} & {h}_{( n - 1 ) m} \\ {h}_{na} & {h}_{nb} & … & {h}_{n ( m - 1 )} & {h}_{nm}\end{matrix} ) ( \begin{matrix}{f}_{a} \\ {f}_{b} \\ ⋮ \\ {f}_{m - 1} \\ {f}_{m}\end{matrix} ) \qquad ( 2 )$$

Note that, in Expression (1), x′₁ to x′ₙ (n is an integer equal to or greater than 1) represent velocity vectors for deriving a state value of a velocity, x′ₐ to x′ₘ (m is an integer equal to or greater than 1) represent vectors whose elements are a reference value and a velocity based on an action of the actuator (a velocity of the movable element of the actuator or a velocity of a member moved by the actuator), and h₁ₐ to hₙₘ represent elements of a transformation matrix representing the function. Furthermore, in Expression (2), f″₁ to f″ₙ (n is an integer equal to or greater than 1) represent force vectors for deriving a state value of force, and f″ₐ to f″ₘ (m is an integer equal to or greater than 1) represent vectors whose elements are a reference value and force based on an action of the actuator (force of the movable element of the actuator or force of a member moved by the actuator).

By setting the coordinate transformation in the function-specific force/velocity distribution transformation block FT in accordance with the function to be implemented, various operations may be implemented and scaling may be performed.

That is, in the basic principle of the present invention, the function-specific force/velocity distribution transformation block FT “transforms” a variable (variable in the real space) of a single actuator to a variable group (variables in the virtual space) of the entire system expressing the function to be implemented, and distributes the control energy to velocity control energy and force control energy. In other words, the basic principle of the present invention transforms a coordinate space in which the velocity and the force are related to each other into a coordinate space in which the velocity and the force are independent from each other, and then performs an arithmetic operation regarding control of the velocity and the force. Thus, as compared with a case where the control is performed by using the variable (variable in the real space) of the single actuator, the velocity control energy and the force control energy may be given independently.

The ideal force origin block FC is a block that performs arithmetic operations in the domain of the force in accordance with coordinate transformation defined by the function-specific force/velocity distribution transformation block FT. The ideal force origin block FC sets a target value related to force in performing an arithmetic operation based on the coordinate transformation defined by the function-specific force/velocity distribution transformation block FT. The target value is set as a fixed value or a variable value in accordance with a function to be implemented. For example, in a case where the function to be implemented is similar to a function indicated by a reference value, the target value can be set to zero, and in a case where scaling is performed, a value obtained by expanding or reducing information indicating the function to be implemented can be set.

The ideal velocity origin block PC is a block that performs an arithmetic operation in the domain of the velocity in accordance with coordinate transformation defined by the function-specific force/velocity distribution transformation block FT. The ideal velocity origin block PC sets a target value related to a velocity in performing an arithmetic operation based on the coordinate transformation defined by the function-specific force/velocity distribution transformation block FT. The target value is set as a fixed value or a variable value in accordance with a function to be implemented. For example, in a case where the function to be implemented is similar to a function indicated by a reference value, the target value can be set to zero, and in a case where scaling is performed, a value obtained by expanding or reducing information indicating the function to be implemented can be set.

The inverse transformation block IFT is a block that transforms values in the domains of the velocity and the force to values in a domain of an input to the control object system S (for example, voltage values, current values, or the like).

According to such a basic principle, when information regarding a position in the actuator of the control object system S is input to the function-specific force/velocity distribution transformation block FT, the function-specific force/velocity distribution transformation block FT uses information regarding a velocity and force obtained on the basis of the information regarding the position to apply the control rules corresponding to a function in each of the domains of the position and the force. Then, an arithmetic operation of force corresponding to the function is performed in the ideal force origin block FC, an arithmetic operation of a velocity corresponding to the function is performed in the ideal velocity origin block PC, and control energy is allocated to each of the force and the velocity.

Arithmetic operation results in the ideal force origin block FC and the ideal velocity origin block PC are information indicating control targets of the control object system S. These arithmetic operation results are used as input values of the actuator in the inverse transformation block IFT and input to the control object system S.

As a result, the actuator of the control object system S executes an operation in accordance with the function defined by the function-specific force/velocity distribution transformation block FT, and the operation of the device that is the object is implemented.

Furthermore, in a case where a tactile force transmission function involving scaling (amplification and reduction of force or a position) is implemented, the coordinate transformation in the function-specific force/velocity distribution transformation block FT in FIG. 2 is represented as the following Expressions (3) and (4).

### [Math. 2]

$$( \begin{matrix}{\dot{x}}_{p} \\ {\dot{x}}_{f}\end{matrix} ) ( \begin{matrix}1 & - α \\ 1 & β\end{matrix} ) ( \begin{matrix}{\dot{x}}_{m} \\ {\dot{x}}_{s}\end{matrix} ) \qquad ( 3 )$$

$$( \begin{matrix}{f}_{p} \\ {f}_{f}\end{matrix} ) = ( \begin{matrix}1 & - α \\ 1 & β\end{matrix} ) ( \begin{matrix}{f}_{m} \\ {f}_{s}\end{matrix} ) \qquad ( 4 )$$

Note that, in Expression (3), x′ₚ is a velocity for deriving a state value of a velocity, and x′_{f} is a velocity related to a state value of force. Furthermore, x′ₘ is a velocity of a reference value (input from the master device 10) (a differential value of a current position of the master device 10), and x′ₛ is a current velocity of the slave device 20 (a differential value of the current position). Furthermore, in Expression (4), fₚ is force related to a state value of a velocity, and f_{f} is force for deriving a state value of force. Furthermore, fₘ is force of a reference value (input from the master device 10), and fₛ is current force of the slave device 20.

In the case of the coordinate transformation indicated in Expressions (3) and (4), a position of the slave device 20 is multiplied by a (a is a positive number) and transmitted to the master device 10, and force of the slave device 20 is multiplied by β (β is a positive number) and transmitted to the master device 10. Then, for example, by setting α=1 and β=1, the tactile force is transmitted without being amplified (that is, enlarged) or attenuated (that is, reduced). On the other hand, for example, by setting the value of a and the value of B according to the purpose, it is possible to realize scaling of amplifying (that is, enlarging) or attenuating (that is, reducing) the transmitted tactile force.

### [Hardware Configuration]

Next, a hardware configuration of a control system in the information presentation system 1 will be described.

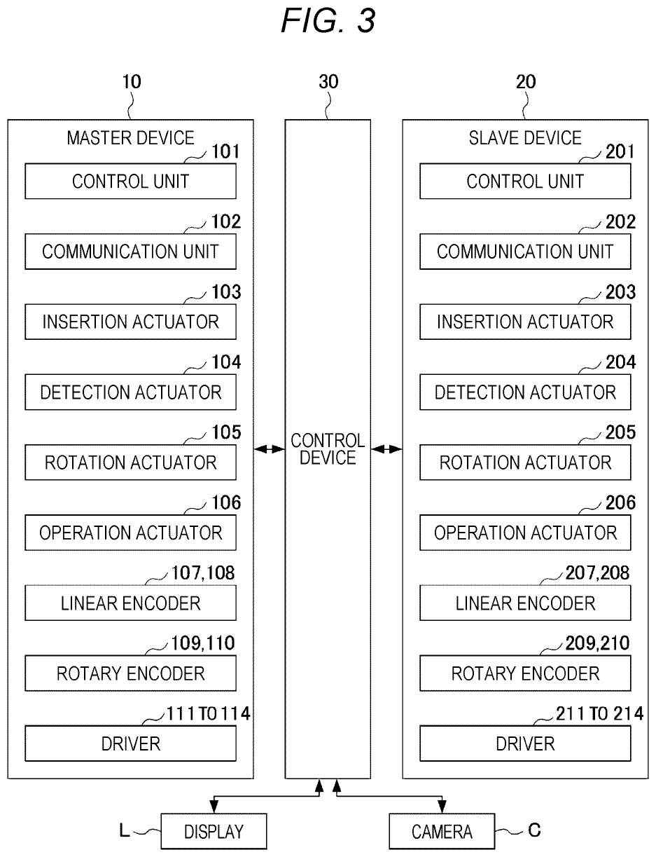

FIG. 3 is a block diagram illustrating the hardware configuration of the control system in the information presentation system 1.

As illustrated in FIG. 3, the information presentation system 1 includes, as the hardware configuration of the control system, the information presentation device 30 including the information processing device such as a PC or a server computer, a control unit 101, a communication unit 102, an insertion actuator 103, a detection actuator 104, a rotation actuator 105, an operation actuator 106, linear encoders 107 and 108, rotary encoders 109 and 110, and drivers 111 to 114 of the master device 10, a control unit 201, a communication unit 202, an insertion actuator 203, a detection actuator 204, a rotation actuator 205, an operation actuator 206, linear encoders 207 and 208, rotary encoders 209 and 210, and drivers 211 to 214 of the slave device 20, the display L, and the cameras C.

The control unit 101 of the master device 10 includes a microcomputer including a processor, a memory, and the like, and controls an operation of the master device 10. For example, the control unit 101 controls driving of the insertion actuator 103, the detection actuator 104, the rotation actuator 105, and the operation actuator 106 of the master device 10 in accordance with control parameters transmitted from the information presentation device 30.

The communication unit 102 controls communication performed by the master device 10 with another device via the network 40.

The insertion actuator 103 includes, for example, a linear motion motor, and applies reaction force to an operation of advancing and retracting the catheter for inserting the catheter into a blood vessel, which is input to the master device 10 by an operator, in accordance with an instruction from the control unit 101.

The detection actuator 104 includes, for example, a voice coil motor, and applies reaction force to an operation of advancing and retracting the catheter for treatment near a lesion, which is input to the master device 10 by an operator, in accordance with an instruction from the control unit 101.

In the present embodiment, the insertion actuator 103 has a longer stroke than the detection actuator 104, while the detection actuator 104 can control a position and force with higher accuracy than the insertion actuator 103.

The rotation actuator 105 includes, for example, a rotary motor, and applies reaction force to an operation of rotating the master device 10 about a rotation axis along an advancing and retracting direction by an operator, in accordance with an instruction from the control unit 101.

The operation actuator 106 includes, for example, a rotary motor, and applies reaction force to an operation input to a lever (grip portion) or the like for operating the end effector by an operator, in accordance with an instruction from the control unit 101.

The linear encoder 107 detects a position (advancing and retracting position on a translation axis) of a movable element of the insertion actuator 103.

The linear encoder 108 detects a position (advancing and retracting position on a translation axis) of a movable element of the detection actuator 104.

The rotary encoder 109 detects a position (rotation angle) of a movable element of the rotation actuator 105.

The rotary encoder 110 detects a position (rotation angle) of a movable element of the operation actuator 106.

The driver 111 outputs a drive current to the insertion actuator 103 in accordance with an instruction from the control unit 101.

The driver 112 outputs a drive current to the detection actuator 104 in accordance with an instruction from the control unit 101.

The driver 113 outputs a drive current to the rotation actuator 105 in accordance with an instruction from the control unit 101.

The driver 114 outputs a drive current to the operation actuator 106 in accordance with an instruction from the control unit 101.

The control unit 201 of the slave device 20 includes a microcomputer including a processor, a memory, and the like, and controls an operation of the slave device 20. For example, the control unit 201 controls driving of the insertion actuator 203, the detection actuator 204, the rotation actuator 205, and the operation actuator 206 of the slave device 20 in accordance with control parameters transmitted from the information presentation device 30.

The communication unit 202 controls communication performed by the slave device 20 with another device via the network 40.

The insertion actuator 203 includes, for example, a linear motion motor, and advances and retracts the catheter of the slave device 20 in accordance with an operation of advancing and retracting the catheter for inserting the catheter into a blood vessel, which is input to the master device 10 by an operator, in accordance with an instruction from the control unit 201.

The detection actuator 204 includes, for example, a voice coil motor, and advances and retracts the catheter of the slave device 20 in accordance with an operation of advancing and retracting the catheter for treatment near a lesion, which is input to the master device 10 by an operator, in accordance with an instruction from the control unit 201.

In the present embodiment, the insertion actuator 203 has a longer stroke than the detection actuator 204, while the detection actuator 204 can control a position and force with higher accuracy than the insertion actuator 203.

The rotation actuator 205 includes, for example, a rotary motor, and rotates the catheter of the slave device 20 about a rotation axis along an advancing and retracting direction in accordance with an operation input to the master device 10 by an operator, in accordance with an instruction from the control unit 201.

The operation actuator 206 includes, for example, a rotary motor, and operates (performs an expansion/contraction operation, an opening/closing operation, and the like on) the end effector in accordance with an operation input to the master device 10 by an operator, in accordance with an instruction from the control unit 201.

The linear encoder 207 detects a position (advancing and retracting position on a translation axis) of a movable element of the insertion actuator 203.

The linear encoder 208 detects a position (advancing and retracting position on a translation axis) of a movable element of the detection actuator 204.

The rotary encoder 209 detects a position (rotation angle) of a movable element of the rotation actuator 205.

The rotary encoder 210 detects a position (rotation angle) of a movable element of the operation actuator 206.

The driver 211 outputs a drive current to the insertion actuator 203 in accordance with an instruction from the control unit 201.

The driver 212 outputs a drive current to the detection actuator 204 in accordance with an instruction from the control unit 201.

The driver 213 outputs a drive current to the rotation actuator 205 in accordance with an instruction from the control unit 201.

The driver 214 outputs a drive current to the operation actuator 206 in accordance with an instruction from the control unit 201.

The display L is installed at a place where an operator of the master device 10 can visually recognize a screen, and displays an image (such as a visible light image or an X-ray image of a subject captured by the camera C) instructed to be displayed by the information presentation device 30, and information instructed to be displayed by the information presentation device 30.

The camera C is installed at a place where the slave device 20 can capture a subject into which the catheter is inserted, captures an image (such as a visible light image or an X-ray image) of the subject, and transmits the captured image to the information presentation device 30.

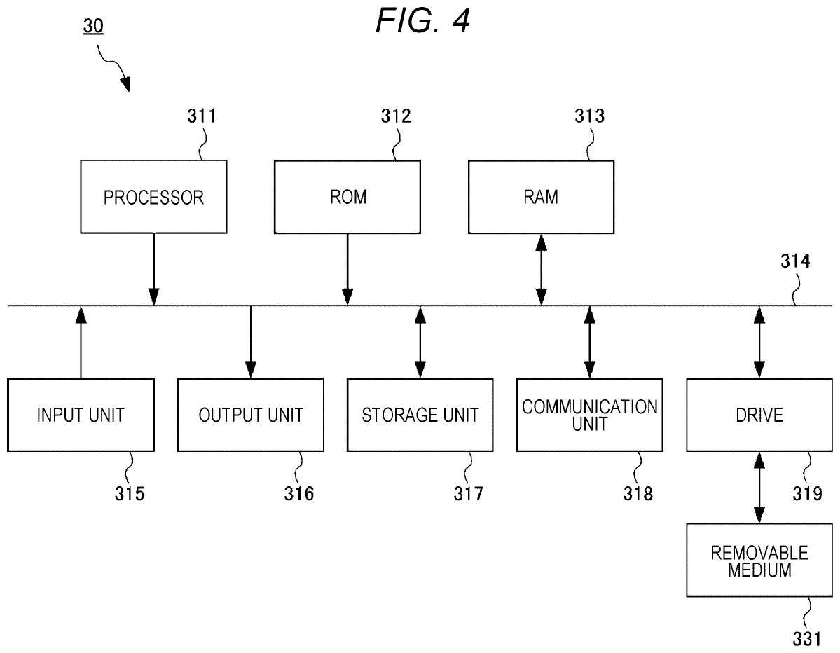

FIG. 4 is a schematic diagram illustrating a hardware configuration of the information processing device constituting the information presentation device 30.

As illustrated in FIG. 4, the information presentation device 30 includes a processor (central processing unit) 311, a read only memory (ROM) 312, a random access memory (RAM) 313, a bus 314, an input unit 315, an output unit 316, a storage unit 317, a communication unit 318, and a drive 319.

The processor 311 executes various types of processing in accordance with a program recorded in the ROM 312 or a program loaded from the storage unit 317 to the RAM 313.

The RAM 313 appropriately stores data and the like necessary for the processor 311 to execute various types of processing.

The processor 311, the ROM 312, and the RAM 313 are mutually connected via the bus 314. The input unit 315, the output unit 316, the storage unit 317, the communication unit 318, and the drive 319 are connected to the bus 314.

The input unit 315 includes various buttons and the like, and inputs various types of information in accordance with an instruction operation.

The output unit 316 includes a display, a speaker, and the like, and outputs an image and sound.

Note that, in a case where the information presentation device 30 is configured as a smartphone or a tablet terminal, the input unit 315 and the display of the output unit 316 may be arranged in an overlapping manner to configure a touch panel.

The storage unit 317 includes a hard disk, a dynamic random access memory (DRAM), or the like, and stores various types of data managed by each server.

The communication unit 318 controls communication performed by the information presentation device 30 with another device via the network.

A removable medium 331 including a magnetic disk, an optical disk, a magneto-optical disk, a semiconductor memory, or the like is appropriately mounted on the drive 319. A program read from the removable medium 331 by the drive 319 is installed in the storage unit 317 as necessary.

### [Functional Configuration]

Next, a functional configuration of the information presentation system 1 will be described.

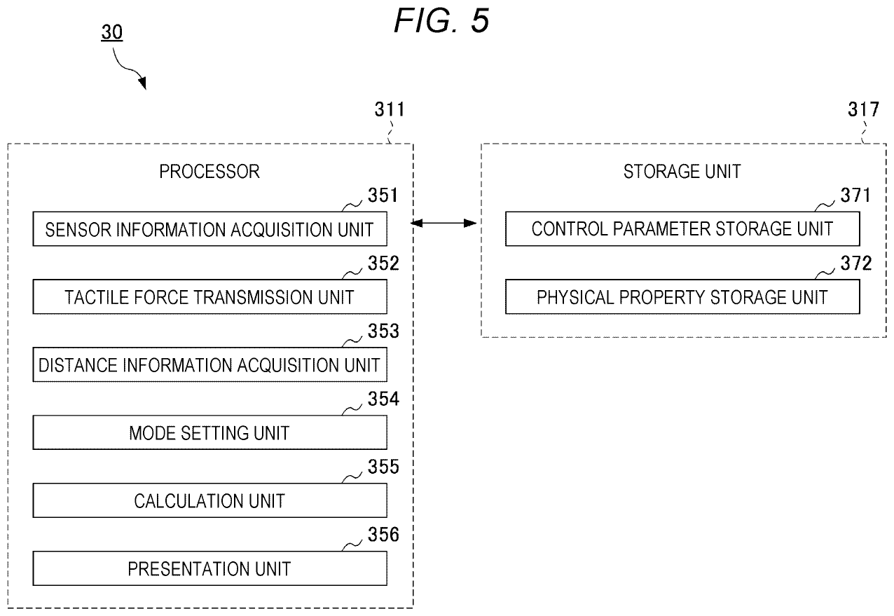

FIG. 5 is a block diagram illustrating the functional configuration of the information presentation system 1.

As illustrated in FIG. 5, in the information presentation system 1, the information presentation device 30 executes various types of processing, whereby a sensor information acquisition unit 351, a tactile force transmission unit 352, a distance information acquisition unit 353, a mode setting unit 354, a calculation unit 355, and a presentation unit 356 function in the CPU 311. Furthermore, a control parameter storage unit 371 and a physical characteristics storage unit 372 are formed in the storage unit 317.

The control parameter storage unit 371 stores, in time series, control parameters acquired in control in which the information presentation device 30 transmits tactile force between the master device 10 and the slave device 20. In the present embodiment, information stored as the control parameters can be various parameters acquired by the tactile force transmission control, and can include various types of information capable of reproducing the tactile force transmission control. For example, sensor information acquired in the master device 10 and the slave device 20, a state value obtained by coordinate transformation of the sensor information, a current command value to each actuator, various setting values set in the information presentation device 30 for the tactile force transmission control, and the like can be stored as the control parameters.

The physical characteristics storage unit 372 stores the physical characteristics of the substance in contact with the slave device 20, the physical characteristics being calculated by the calculation unit 355. In the present embodiment, the calculation unit 355 calculates “elasticity”, “viscosity”, and “inertia” as the physical characteristics of the substance. Therefore, the physical characteristics storage unit 372 stores each of “elasticity”, “viscosity”, and “inertia”.

Furthermore, in the present embodiment, the presentation unit 356 presents the physical characteristics of the substance calculated by the calculation unit 355 and the physical characteristics of the reference substance in a comparable manner. Therefore, the physical characteristics storage unit 372 also stores the physical characteristics of the reference substance. The physical characteristics of the reference substance may be physical characteristics calculated by the calculation unit 355 in the past, or may be physical characteristics measured by a test method or a tactile method different from the calculation by the calculation unit 355.

The sensor information acquisition unit 351 acquires sensor information detected by various sensors installed in the master device 10 and the slave device 20. For example, the sensor information acquisition unit 351 acquires information indicating the positions (the advancing and retracting positions or the rotation angles) of the movable elements of the actuators detected by the linear encoders 107, 108, 207, and 208 and the rotary encoders 109, 110, 209, and 210. Furthermore, the sensor information acquisition unit 351 stores the acquired sensor information in the control parameter storage unit 371 as time-series data.

The tactile force transmission unit 352 controls tactile force transmission in the master device 10 and the slave device 20 in accordance with the control algorithm illustrated in FIG. 2. For example, the tactile force transmission unit 352 executes control to transmit tactile force between the actuators for corresponding operations of the master device 10 and the slave device 20 in the information presentation processing.

The distance information acquisition unit 353 acquires distance information that is information indicating a distance between the distal end of the catheter and the lesion by performing calculation, analysis, and the like on various data. Further, the distance information acquisition unit 353 outputs the acquired distance information to the mode setting unit 354. This distance information is used by the mode setting unit 354 to determine whether or not to switch the mode.

The distance information acquisition unit 353 acquires the distance information using an image captured by the camera C. In this case, for example, the distance information acquisition unit 353 acquires the distance information by analyzing this image and calculating the distance between the distal end of the catheter and the lesion. By thus calculating the distance between the distal end of the catheter and the lesion from the image captured by the camera C, it is possible to determine approach of the catheter to the lesion on the basis of a criterion similar to that of a case where a human visually determines the approach.

In addition, the distance information acquisition unit 353 may acquire the distance information using various sensors. In this case, for example, the distance information is acquired by providing a marker for magnetic detection at the distal end of the catheter, detecting the position of the catheter by a magnetic sensor from the outside of the subject, and calculating the distance to the lesion. Alternatively, the distance information acquisition unit 353 may acquire the distance information by installing a sensor for detecting a distal end position of the catheter in advance inside the subject, detecting the position of the catheter is by the sensor, and calculating the distance to the lesion.

The distance information acquisition unit 353 outputs the distance information thus acquired to the mode setting unit 354.

The mode setting unit 354 switches and sets three modes: an “insertion mode”; a “detection mode”; and a “measurement mode”, as modes set in the information presentation device 30.

Specifically, on the basis of the distance between the distal end of the catheter and the lesion acquired as the distance information by the distance information acquisition unit 353, the mode setting unit 354 switches to the “insertion mode” in a case where the distal end of the catheter has not reached the vicinity of the lesion, and switches to the “detection mode” in a case where the distal end of the catheter has reached the vicinity of the lesion. In this case, the standard of reduction in the distance for switching the mode (that is, a threshold value related to a distance for determining whether or not to switch) can be set on the basis of, for example, a measured value, a statistical value, an estimated value obtained by simulation, or the like when the catheter was inserted into the subject or a biological model imitating the subject in the past.

In addition, the mode setting unit 354 switches between the “detection mode” and the “measurement mode” on the basis of a mode switching operation by the operator via the input unit 315 or a mode switching operation by communication from an external device (for example, the master device 10) via the communication unit 318. That is, the operator can switch between the “detection mode” and the “measurement mode” at any timing desired by the operator.

The “insertion mode” is a mode in which the insertion actuator 203 is used to advance and retract the catheter in the slave device 20 and a tactile force is transmitted to/from the insertion actuator 103 of the master device 10. The “insertion mode” is, for example, a mode set before an operator inserts the catheter into a subject and a distal end of the catheter reaches near a lesion.

The “detection mode” is a mode in which the detection actuator 204 is used to advance and retract the catheter in the slave device 20 and a tactile force is transmitted to/from the detection actuator 104 of the master device 10. The “detection mode” is, for example, a mode set after an operator inserts the catheter into a subject and the distal end of the catheter reaches near a lesion.

The “measurement mode” is a mode in which the detection actuator 204 is used to advance and retract the catheter in the slave device 20, and physical characteristics of a substance in contact with the catheter is calculated and presented with the advancing and retracting. Unlike the “insertion mode” and the “detection mode”, the “measurement mode” does not accept an operation by the operator in the master device 10. Then, in the “measurement mode”, the slave device 20 does not perform an operation corresponding to the operation on the master device 10, but performs an operation to maintain a predetermined motion state by driving the detection actuator 204 in accordance with the instruction for realizing the predetermined motion state generated by the tactile force transmission unit 352. The “measurement mode” is, for example, a mode set to present the physical characteristics of a substance in a lesion to the operator or the like.

As described above, in the present embodiment, the insertion actuator 103, 203 has a longer stroke than the detection actuator 104, 204, while the detection actuator 104, 204 can control a position and force with higher accuracy than the insertion actuator 103, 203. Therefore, the “detection mode” is used in a situation where the operator needs to feel a minute external force input to the slave device 20 in contrast to the “insertion mode”. Furthermore, the “measurement mode” is used in a state where it is necessary to accurately calculate the physical characteristics of the substance in the lesion on the basis of the minute external force input to the slave device 20.

When the “measurement mode” is set by the mode setting unit 354, the calculation unit 355 calculates physical characteristics of the substance in contact with the slave device 20 (here, the substance in the lesion in contact with the distal end of the catheter). In order to calculate these physical characteristics of the substance, in the “measurement mode”, the tactile force transmission unit 352 generates an instruction for achieving a predetermined motion state. The slave device 20 performs an operation to maintain the predetermined motion state by driving the detection actuator 204 in accordance with the instruction for realizing the predetermined motion state generated by the tactile force transmission unit 352.

On the other hand, while the predetermined motion state is maintained, the calculation unit 355 acquires values indicating various external forces input from the environment to the slave device 20 (hereinafter, referred to as a “force value”) by the operation of the slave device 20. This force value can be calculated as a product of mass and acceleration. Therefore, the distance information acquisition unit 353 acquires the force value by performing calculation such as integration on a real-time basis on the basis of the sensor information acquired by the sensor information acquisition unit 351 and stored as the control parameter in the control parameter storage unit 371 and the information corresponding to the result of the coordinate transformation performed by the function-specific force/velocity distribution transformation block FT in the control algorithm illustrated in FIG. 2. Note that, in this case, the distance information acquisition unit 353 may acquire the force value after performing filter processing using a band-limiting filter on the waveform of the instantaneous value. In this manner, the calculation unit 355 can calculate the force value on the basis of the control parameter. Therefore, in the present embodiment, it is not necessary to provide a force sensor for measuring the force value.

Here, as described above, the calculation unit 355 calculates elasticity, viscosity, and inertia as the physical characteristics of the substance. A method of calculating each of these physical characteristics by the calculation unit 355 and control performed for the calculation by the tactile force transmission unit 352 will be described below.

In a case where the calculation unit 355 calculates elasticity, the predetermined motion state is a state in which the catheter is inserted by uniform motion. Therefore, the tactile force transmission unit 352 calculates the velocity (or angular velocity) by differentiating, on a real-time basis, the position (or angle) of the movable portion (the movable element of the actuator, the catheter moved by the actuator, or the like) moved by the operation of the slave device 20, which is acquired as the sensor information. Then, the tactile force transmission unit 352 controls the operation of the slave device 20 such that the calculated velocity (or angular velocity) becomes a constant value. Furthermore, the tactile force transmission unit 352 continues this control until the movable portion moved by the operation of the slave device 20 moves by a predetermined travel distance (Δx). As a result, the uniform motion, that is the predetermined motion state, is maintained. On the other hand, the calculation unit 355 subtracts the force value at the start of the uniform motion from the force value at the end of the uniform motion to calculate the amount of change in force (ΔF). Then, the calculation unit 355 calculates a value obtained by dividing the amount of change in force (ΔF) by the travel distance (Δx) while the uniform motion is maintained as a value indicating the elasticity (corresponding to the spring constant) of the substance (here, the material in the lesion in contact with the distal end of the catheter) in contact with the slave device 20.

In a case where the calculation unit 355 calculates viscosity, the predetermined motion state is a state in which the catheter is inserted by uniform acceleration motion. Therefore, the tactile force transmission unit 352 calculates the velocity (or angular velocity) in a similar manner to the case of calculating the elasticity. Then, the tactile force transmission unit 352 further differentiates the calculated velocity (or angular velocity) to calculate an acceleration (or angular acceleration). Then, the tactile force transmission unit 352 controls the operation of the slave device 20 such that the calculated acceleration (or angular acceleration) becomes a constant value. Furthermore, the tactile force transmission unit 352 continues this control until the movable portion moved by the operation of the slave device 20 moves by a predetermined travel distance (Δx). As a result, the uniform acceleration motion, that is the predetermined motion state, is maintained. On the other hand, the calculation unit 355 subtracts the force value at the start of the uniform acceleration motion from the force value at the end of the uniform acceleration motion to calculate the amount of change in force (ΔF). Then, the calculation unit 355 calculates a value obtained by dividing the amount of change in force (ΔF) by the travel distance (Δx) while the uniform acceleration motion is maintained as a value indicating the viscosity of the substance (here, the material in the lesion in contact with the distal end of the catheter) in contact with the slave device 20.

In a case where the calculation unit 355 calculates inertia, the predetermined motion state is a state in which the catheter is inserted by uniform jerk motion (also referred to as uniform jolt motion). Therefore, the tactile force transmission unit 352 calculates the acceleration (or angular acceleration) in a similar manner to the case of calculating the viscosity. Then, the tactile force transmission unit 352 further differentiates the calculated acceleration (or angular acceleration) to calculate a jerk (or angular jerk). Then, the tactile force transmission unit 352 controls the operation of the slave device 20 such that the calculated jerk (or angular jerk) becomes a constant value. Furthermore, the tactile force transmission unit 352 continues this control until the movable portion moved by the operation of the slave device 20 moves by a predetermined travel distance (Δx). As a result, the uniform jerk motion, that is the predetermined motion state, is maintained. On the other hand, the calculation unit 355 subtracts the force value at the start of the uniform jerk motion from the force value at the end of the uniform jerk motion to calculate the amount of change in force (ΔF). Then, the calculation unit 355 calculates a value obtained by dividing the amount of change in force (ΔF) by the travel distance (Δx) while the uniform jerk motion is maintained as a value indicating the inertia of the substance (here, the material in the lesion in contact with the distal end of the catheter) in contact with the slave device 20.

In this manner, the elasticity, viscosity, and inertia calculated by the calculation unit 355 on the basis of the force value under the quantitative state in which the slave device 20 maintains the predetermined motion state are stored in the physical characteristics storage unit 372. Note that the calculation unit 355 does not necessarily need to calculate all of the elasticity, viscosity, and inertia, and may calculate at least one of them.

The presentation unit 356 presents physical characteristics such as elasticity, viscosity, and inertia calculated by the calculation unit 355 to the operator and a person who performs various analyses, tests, and the like. The presentation can be realized, for example, by displaying a numerical value, a graph, or the like indicating the physical characteristics or the force value on the display L. Alternatively, the presentation can also be realized by outputting a numerical value or the like by sound from a speaker included in the output unit 316.

The timing at which the presentation is performed by the presentation unit 356 is, for example, performed on a real-time basis at the same time as the calculation unit 355 calculates the physical characteristics in the “measurement mode”. In addition, thereafter, also in a case where the mode is switched again to the “detection mode” or the “insertion mode”, the presentation may be continued. Alternatively, after the end of the information presentation processing, presentation may be performed in order to perform various analyses, tests, and the like.

Furthermore, during the presentation, the presentation unit 356 may present the physical characteristics of the substance calculated by the calculation unit 355 and the physical characteristics of the reference substance in a comparable manner. As described above, the reference physical characteristics are stored in the physical characteristics storage unit 372. The physical characteristics of the reference substance may be physical characteristics calculated by the calculation unit 355 in the past, or may be physical characteristics measured by a test method or a tactile method different from the calculation by the calculation unit 355. The physical characteristics of the reference substance are, for example, standard values such as elasticity of a lesion when the lesion is calcified. The operator or the like can grasp the state of the lesion (here, the degree of calcification) by comparing the standard values of the lesion in such a specific state with the values calculated by the presentation unit 356.

By referring to the presented physical characteristics, the operator or the like can grasp the state of the lesion or the like by an objective index such as a numerical value. That is, it is possible to grasp both the subjective index, which is the transmitted tactile force, and the objective index. As a result, it is possible to provide further assistance in addition to assistance by tactile force transmission.

### [Operation]

Next, an operation of the information presentation system 1 will be described.

### [Information Presentation Processing]

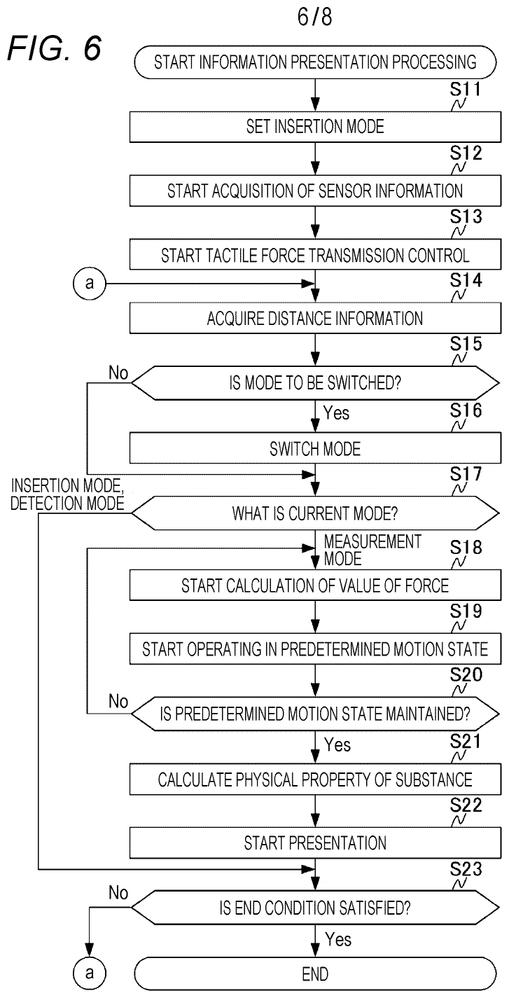

FIG. 6 is a flowchart for describing a flow of information presentation processing executed by the information presentation device 30.

The information presentation processing is started in response to an instruction to execute the information presentation processing from the operator via the input unit 315 or an instruction to execute the information presentation processing by communication from an external device (for example, the master device 10) via the communication unit 318. In the present embodiment, in a case where the information presentation processing is started, it is assumed that the processing is started in a state where the distal end of the catheter is inserted into a subject by a predetermined distance (for example, a state where about 1 to 10 [cm] is inserted) manually by an assistant assisting an operation of the slave device 20 or by a remote operation from the master device 10. With this configuration, it is possible to suppress control of the information presentation device 30 from becoming unstable in a state where a change in external force at an initial stage of the insertion is large.

In step S11, the mode setting unit 354 sets the insertion mode.

In step S12, the sensor information acquisition unit 351 starts acquiring the sensor information detected by various sensors installed in the master device 10 and the slave device 20. This acquisition of the sensor information is performed in parallel with other steps until this processing is completed. The acquired sensor information is stored in the control parameter storage unit 371 as the time-series data.

In step S13, the tactile force transmission unit 352 starts control of tactile force transmission based on the sensor information. The control of the tactile force transmission is performed in parallel with other steps until the present processing is completed.

In step S14, the distance information acquisition unit 353 acquires the distance information by performing calculation, analysis, and the like on various data. Further, the distance information acquisition unit 353 outputs the acquired distance information to the mode setting unit 354.

In step S15, the mode setting unit 354 determines whether or not to switch the mode on the basis of the distance information and the mode switching operation from the operator. That is, it is determined whether or not to switch between the “insertion mode” and the “detection mode” on the basis of the distance between the distal end of the catheter and the lesion indicated by the distance information, and whether or not to switch between the “detection mode” and the “measurement mode” on the basis of the presence or absence of the mode switching operation by the operator. In a case of switching to any mode, determination in step S15 is Yes, and the processing proceeds to step S16. On the other hand, when the mode is not to be switched, determination in step S15 is No, and the processing proceeds to step S17.

In step S16, the mode setting unit 354 switches the mode. That is, the mode is switched and set to any one of the “insertion mode”, the “detection mode”, and the “measurement mode” in accordance with the determination result of step S15.

In step S17, the calculation unit 355 determines whether the currently set mode is the “insertion mode” or the “detection mode”, or the “measurement mode”. In a case of the “insertion mode” or the “detection mode”, determination in step S17 is “detection mode, insertion mode”, and the processing proceeds to step S23. On the other hand, in the case of the “measurement mode”, determination in step S17 is “measurement mode”, and the processing proceeds to step S18.

In step S18, the calculation unit 355 starts calculation of the force value.

In step S19, the tactile force transmission unit 352 starts control of the operation of the slave device 20 so as to be in a predetermined motion state.

In step S20, the tactile force transmission unit 352 determines whether or not the movable portion moved by the operation of the slave device 20 has moved by the predetermined travel distance (Δx). In a case where the movable portion has moved by the predetermined travel distance (Δx), determination in step S20 is Yes, and the processing proceeds to step S21. On the other hand, in a case where the movable portion has not moved by the predetermined travel distance (Δx), determination in step S20 is No, and the processing returns to step S18 and is repeated.

In step S21, the calculation unit 355 calculates the physical characteristics of the substance (here, the substance in the lesion in contact with the distal end of the catheter).

In step S22, the presentation unit 356 starts presentation of the physical characteristics calculated in step S21.

Note that, in a case where the mode is switched from “measurement mode” to the “detection mode” and then to “measurement mode” again, or in a case where the operator does not perform the switching operation from the “measurement mode” to the “detection mode”, the processing of steps S18 to S22 is repeated a plurality of times. In this case, a certain physical characteristic (for example, elasticity) may be repeatedly calculated, and a change in the calculated value may be presented in the form of a graph, or elasticity, viscosity, and inertia may be calculated and presented in order.

In step S23, the tactile force transmission unit 352 determines whether or not an end condition that is a condition for ending the present processing is satisfied. The end condition is, for example, that an end instruction operation of the information presentation processing via the input unit 315 from the operator, or an end instruction of the information presentation processing by communication from an external device (for example, the master device 10) via the communication unit 318 is given. In a case where the end condition is satisfied, determination in step S23 is Yes, and this process ends. On the other hand, in a case where the end condition is not satisfied, determination in step S23 is No, and the processing returns to step S14 and is repeated.

The information presentation processing described above can present the physical characteristics of the substance in contact with the slave device 20 on the basis of an external force input from the environment to the slave device 20 in a quantitative state in which the slave device 20 maintains a predetermined motion state. Therefore, for example, the physical characteristics of a substance such as a blood vessel that cannot be directly touched by the operator can be quantitatively presented as information (that is, the tactile information) obtained by the slave device 20 in contact with the substance, whereby it is possible to further assist the operation of the operator. In addition to the operation by the operator, it is possible to further assist a person who performs various analyses, tests, and the like using the presented physical characteristics.

Therefore, according to the information presentation processing, it is possible to solve the problem of providing further assistance in addition to assistance by tactile force transmission.

### [Comparison Regarding External Force]

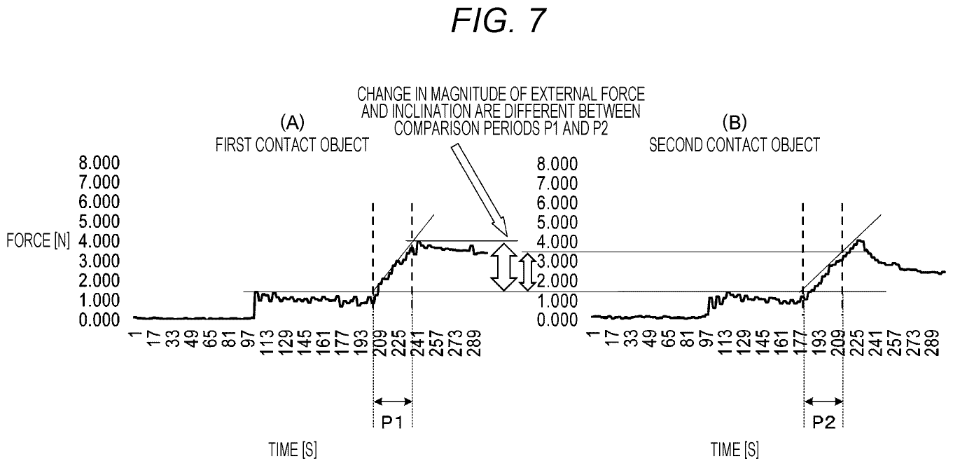

FIG. 7 is a schematic diagram illustrating a time-series change in an external force input from the environment to a slave device 20 when the slave device 20 comes into contact with a substance in the above-described embodiment.

FIG. 7 (A) is a schematic diagram illustrating a case where the slave device 20 comes into contact with an acrylic wall as a first contact object. FIG. 7 (B) is a schematic diagram illustrating a case where the slave device 20 comes into contact with a blood vessel having arteriosclerosis as a second contact object. In each of FIGS. 7 (A) and 7 (B), the abscissa represents time [S], and the ordinate represents the force value [N] indicating the magnitude of the external force input to the slave device 20.

Focusing on FIG. 7 (A), the catheter starts moving with the start of the operation, and the force value gradually increases. Then, at the timing where the distal end of the catheter comes into contact with the first contact object, the abutment force with the first contact object increases, and the force value sharply increases.

Then, when the force value increases to some extent, the force value then transitions at a substantially constant magnitude.

Here, a period from the timing at which the force value begins to sharply increase to the timing at which the force value reaches a certain constant magnitude is defined as a comparison period P1.

Next, focusing on FIG. 7 (B), which is generally similar to FIG. 7 (A), the catheter starts moving with the start of the operation, and the force value gradually increases. Then, at the timing where the distal end of the catheter comes into contact with the second contact object, the abutment force with the second contact object increases, and the force value sharply increases.

Then, when the force value increases to some extent, the force value then transitions at a substantially constant magnitude.

Here, a period from the timing at which the force value begins to sharply increase to the timing at which the same length of time as the comparison period P1 elapses is defined as a comparison period P2.

Comparing the comparison period P1 with the comparison period P2, it can be observed that the change in the force value (that is, the amount of change obtained by subtracting the force value at the start of the comparison period from the force value at the end of the comparison period) is larger in the comparison period P1 than in the comparison period P2. This can also be observed from the fact that the inclination of the graph is larger in the comparison period P1 than in the comparison period P2.

From this comparison, it is obvious that the fact that different contacted substances have different physical characteristics is reflected in the change in force value. Therefore, as in the above-described embodiment, it is possible to calculate the physical characteristics of the contacted substance on the basis of the change in the force value.

### [First Modification]

In the embodiment described above, the configuration has been described as an example in which the actuator operates after the catheter is inserted until the catheter reaches a lesion, but the present invention is not limited thereto. For example, the catheter may be manually inserted to the vicinity of the lesion, the insertion using the master device 10 and the slave device 20 may be started in a specific section in the vicinity of the lesion, the tactile force transmission may be controlled to operate with the actuator similarly to the case of setting the “detection mode”, and the information presentation processing may be performed to switch the mode to the “measurement mode”.

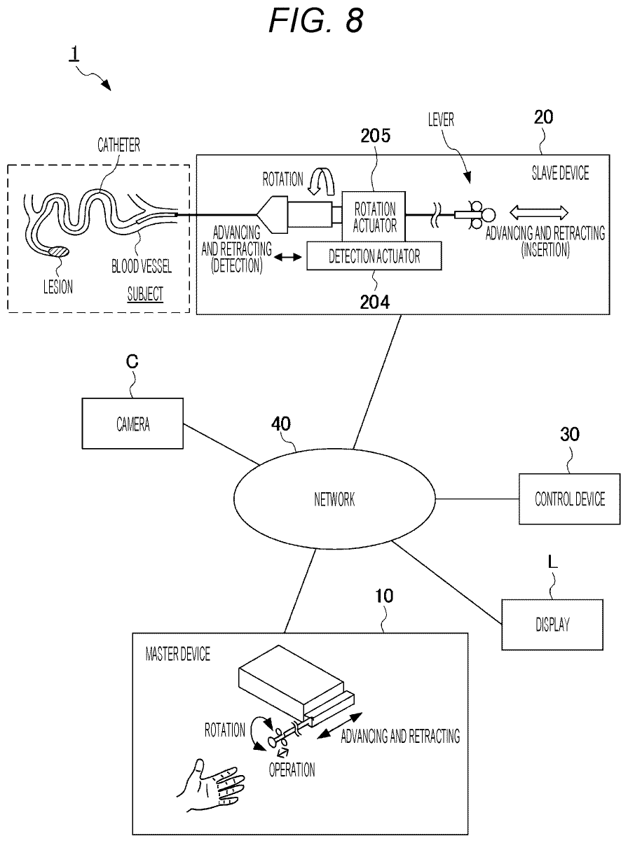

FIG. 8 is a schematic diagram illustrating a configuration of the information presentation system 1 that performs information presentation processing after an operator manually inserts a catheter of the slave device 20.

As illustrated in FIG. 8, in the information presentation system 1 of the present modification, a lever (grip portion) or the like for operation is installed in the catheter of the slave device 20, and a manual operation by an operator is possible.

Furthermore, the information presentation system 1 of the present modification includes only the detection actuators 104 and 204 and does not include the insertion actuators 103 and 203 among the linear motion actuators included in the information presentation system 1 of the first embodiment illustrated in FIG. 1.

In a case where the operator manually inserts the catheter, in the slave device 20, the catheter is released from movement control by the detection actuator 204 and the rotation actuator 205, and can be operated in a similar manner to the conventional catheter.

At this time, it is assumed that the catheter is inserted to a position in front of near the lesion by the operator, and the information presentation processing is started with this state as an initial state.

In a case where the information presentation processing is started, the catheter is held for movement control by the detection actuator 204 and the rotation actuator 205, the slave device 20 moves the catheter in accordance with an operation on the master device 10, and control for transmitting the tactile force by the information presentation device 30 is started. Then, similarly to the case where the “detection mode” is set in the above-described embodiment, the actuator is operated, and the information presentation processing is performed so that the mode can be switched to the “measurement mode”, whereby the physical characteristics of the substance calculated on the basis of the force value can be presented also in the present modification.

According to the present modification, since a distance for moving the catheter by the actuator is relatively short, it is sufficient to provide an actuator having a short stroke such as a voice coil motor, whereby the size and weight of the master device 10 and the slave device 20 can be reduced.

### [Other Modifications]

In the embodiment described above, the description has been made assuming that the force in the thrust direction (advancing and retracting direction) of the catheter is subjected to tactile force transmission between the master device 10 and the slave device 20, but the present invention is not limited thereto. For example, the force related to the rotation about the rotation axis along the advancing and retracting direction or the operation of the end effector may be subjected to the tactile force transmission between the master device 10 and the slave device 20. Furthermore, for example, in the above-described embodiment, the case where the catheter is remotely operated by the information presentation system 1 has been described as an example, but the present invention is not limited thereto. That is, various devices can be employed as the device to be remotely operated by the information presentation system 1, and for example, various devices having a linear portion, for example, medical devices such as forceps or an endoscope can be employed.

Furthermore, in the embodiment described above, the case has been described as an example where the actuator included in the master device 10 and the actuator included in the slave device 20 are associated on a one-to-one basis to perform tactile force transmission, but the present invention is not limited thereto. That is, a plurality of actuators of the master device 10 can be associated with one actuator of the slave device 20 to transmit tactile force, or one actuator of the master device 10 can be associated with a plurality of actuators of the slave device 20 to transmit tactile force. Furthermore, a plurality of actuators of the master device 10 can be associated with a plurality of actuators of the slave device 20 to transmit tactile force. As an example, the insertion actuator 203 and the detection actuator 204 of the slave device 20 illustrated in FIG. 3 can be associated with the insertion actuator 103 of the master device 10 to transmit tactile force. In this case, it is not necessary to provide the detection actuator 104 of the master device 10, and cost reduction, weight reduction of the device, and the like can be implemented.

Furthermore, in the embodiment described above, the configuration including the insertion actuator 203 and the detection actuator 204 as the actuators for advancing and retracting the catheter of the slave device 20 has been described as an example, but the present invention is not limited thereto. That is, the catheter of the slave device 20 may be advanced and retracted by one actuator as long as the actuator satisfies the required performance in a stroke and accuracy of an operation. In this case, the processing of switching between the insertion mode and the detection mode is omitted, and similarly to the case where the “detection mode” is set in the above-described embodiment, the actuator is operated, and the information presentation processing is performed so that the mode can be switched to the “measurement mode”, whereby the physical characteristics of the substance calculated on the basis of the force value can be presented also in the present modification.

In addition, in the above-described embodiment, it has been described that, on the basis of the distance between the distal end of the catheter and the lesion acquired as the distance information by the distance information acquisition unit 353, the mode setting unit 354 switches to the “insertion mode” in a case where the distal end of the catheter has not reached the vicinity of the lesion, and switches to the “detection mode” in a case where the distal end of the catheter has reached the vicinity of the lesion; however, the present invention is not limited thereto. Here, in a case where the catheter is inserted into the subject, for example, various external forces input from the environment change between a state in which the catheter advances in the artery and a state in which the catheter reaches a lesion in a narrowed heart or the like, and accordingly, the force value also changes. Therefore, the mode setting unit 354 calculates the force value in the same manner as the calculation unit 355, determines the insertion state of the catheter on the basis of the force value, and switches between the “insertion mode” and the “detection mode”. In this case, for example, the mode setting unit 354 switches to the “insertion mode” in a case where the force value is less than the predetermined value, and switches to the “detection mode” in a case where the force value becomes greater than or equal to the predetermined value.

In addition, in the above-described embodiment, it has been described that the mode setting unit 354 switches between the “detection mode” and the “measurement mode” on the basis of a mode switching operation by the operator via the input unit 315 or a mode switching operation by communication from an external device (for example, the master device 10) via the communication unit 318; however, the present invention is not limited thereto. For example, if it is possible to accurately determine whether or not the distal end of the catheter has reached the vicinity of the lesion on the basis of the distance information or the like, the mode setting unit 354 may switch between the “detection mode” and the “measurement mode” on the basis of the distance information or the like without requiring the switching operation from the operator or the like. For example, in a case where it is determined that the distal end of the catheter has got closer to the lesion on the basis of the distance information or the like after switching to the “detection mode”, the mode setting unit 354 may switch to the “measurement mode” without requiring the switching operation from the operator or the like. Then, when the physical characteristics of the substance are calculated and presented in the “measurement mode”, the mode setting unit 354 may switch to the “detection mode” without requiring the switching operation from the operator or the like.

### Configuration Example

As described above, the information presentation system 1 according to the present embodiment includes the master device 10 to which the operation by the operator is input and the slave device 20 that operates in accordance with the operation input to the master device 10. Furthermore, the information presentation system 1 includes the tactile force transmission unit 352, the calculation unit 355, and the presentation unit 356.

The tactile force transmission unit 352 controls tactile force transmission in the master device 10 and the slave device 20.

The calculation unit 355 calculates physical characteristics of a substance in contact with the slave device 20 on the basis of an external force input from the environment to the slave device 20 while the slave device 20 maintains a predetermined motion state.

The presentation unit 356 presents the physical characteristics of the substance calculated by the calculation unit 355.

As described above, the information presentation system 1 can present the physical characteristics of the substance in contact with the slave device 20 on the basis of an external force input from the environment to the slave device 20 in a quantitative state in which the slave device 20 maintains a predetermined motion state. Therefore, for example, the physical characteristics of a substance such as a blood vessel that cannot be directly touched by the operator can be quantitatively presented as information (that is, the tactile information) obtained by the slave device 20 in contact with the substance, whereby it is possible to further assist the operation of the operator. In addition to the operation by the operator, it is possible to further assist a person who performs various analyses, tests, and the like using the presented physical characteristics.

Therefore, according to the information presentation system 1, it is possible to solve the problem of providing further assistance in addition to assistance by tactile force transmission.

When the slave device 20 maintains the state of uniform motion as the predetermined motion state, the calculation unit 355 calculates elasticity as the physical characteristics of the substance.

This makes it possible to quantitatively present the elasticity of the substance such as a blood vessel that cannot be directly touched by the operator.

When the slave device 20 maintains the state of uniform acceleration motion as the predetermined action state, the calculation unit 355 calculates viscosity as the physical characteristics of the substance.

This makes it possible to quantitatively present the viscosity of the substance such as a blood vessel that cannot be directly touched by the operator.

When the slave device 20 maintains the state of uniform jerk motion as the predetermined action state, the calculation unit 355 calculates inertia as the physical characteristics of the substance.

This makes it possible to quantitatively present the inertia of the substance such as a blood vessel that cannot be directly touched by the operator.

The calculation unit 355 determines whether or not the slave device 20 maintains the predetermined motion state on the basis of the travel distance by which the slave device 20 has moved since the start of the predetermined motion state.

This makes it possible to determine whether or not the predetermined motion state is maintained using the sensor information for controlling the tactile force transmission.

The presentation unit 356 presents the physical characteristics of the substance calculated by the calculation unit 355 and the physical characteristics of the reference substance in a comparable manner.

As a result, for example, it is possible to compare the quantitative physical characteristics based on the information obtained by the slave device 20 coming into contact with the substance (that is, the tactile information) with the physical characteristics serving as a reference, and to further assist the operator or the like.

The presentation unit 356 presents the physical characteristics of the substance calculated by the calculation unit 355 to the operator who is operating the master device 10.

This makes it possible to assist the operating operator on a real-time basis.

As described above, the information presentation device 30 according to the present embodiment includes the tactile force transmission unit 352, the calculation unit 355, and the presentation unit 356.

The tactile force transmission unit 352 controls the tactile force transmission in the master device 10 to which the operation by the operator is input and the slave device 20 that operates in accordance with the operation input to the master device 10.

The calculation unit 355 calculates physical characteristics of a substance in contact with the slave device 20 on the basis of an external force input from the environment to the slave device 20 while the slave device 20 maintains a predetermined motion state.

The presentation unit 356 presents the physical characteristics of the substance calculated by the calculation unit 355.

With such a configuration of the information presentation device 30, similarly to the above-described information presentation system 1, it is possible to solve the problem of providing further assistance in addition to the assistance by the tactile force transmission.

Note that the present invention is not limited to the embodiment described above, and modification, improvement, and the like within a range in which the object of the present invention can be achieved are included in the present invention.

For example, the present invention can be implemented not only as the information presentation system 1 in the embodiment described above, but also as an information presentation device that controls the information presentation system 1, an information presentation method including each step executed in the information presentation system 1, or a program executed by a processor to implement the functions of the information presentation system 1.

Furthermore, in the embodiment described above, the configuration has been described as an example in which the information presentation device 30 is implemented as an independent device, but the functions of the information presentation device 30 can be mounted on one of the control unit 101 of the master device 10 and the control unit 201 of the slave device 20, or can be mounted in a distributed manner on both of them.

Furthermore, the processing in the embodiment described above can be executed by either hardware or software.

That is, it is sufficient that the information presentation system 1 has a function capable of executing the processing described above, and what functional configuration and hardware configuration are used to implement this function is not limited to the examples described above.

In a case where the processing described above is executed by software, a program constituting the software is installed in a computer from a network or a storage medium.

The storage medium storing the program includes a removable medium distributed separately from the device main body, a storage medium incorporated in the device main body in advance, or the like. The removable medium includes, for example, a semiconductor memory, a magnetic disk, an optical disk, a magneto-optical disk, or the like. The optical disk includes, for example, a compact disk-read only memory (CD-ROM), a digital versatile disk (DVD), a Blu-ray Disc (registered trademark), or the like. The magneto-optical disk includes a mini-disk (MD) or the like. Furthermore, the storage medium incorporated in the device main body in advance includes, for example, a read only memory (ROM) or a hard disk in which the program is stored, a semiconductor memory, or the like.

Note that the embodiment described above indicates an example to which the present invention is applied, and does not limit a technical scope of the present invention. That is, the present invention can be subjected to various changes such as omission and replacement without departing from the gist of the present invention, and various embodiments other than the embodiment described above can be taken. Various embodiments and modification thereof that can be taken by the present invention are included in the invention described in the claims and an equivalent scope thereof.

### REFERENCE SIGNS LIST

1 Information presentation system
10 Master device
20 Slave device
30 Information presentation device
40 Network
L Display
C Camera
FT Function-specific force/velocity distribution transformation block
FC Ideal force origin block
PC Ideal velocity (position) origin block
IFT Inverse transformation block
S Control object system
101, 201 Control unit
102, 202 Communication unit
103, 203 Insertion actuator
104, 204 Detection actuator
105, 205 Rotation actuator
106, 206 Operation actuator
107, 108, 207, 208 Linear encoder
109, 110, 209, 210 Rotary encoder
111 to 114, 211 to 214 Driver
311 Processor
312 ROM
313 RAM
314 Bus
315 Input unit
316 Output unit
317 Storage unit
318 Communication unit
319 Drive
331 Removable medium
351 Sensor information acquisition unit
352 Tactile force transmission unit
353 Distance information acquisition unit
354 Mode setting unit
355 Calculation unit
356 Presentation unit
371 Control parameter storage unit
372 Physical characteristics storage unit

1. An information presentation system including a master device to which an operation by an operator is input and a slave device that operates in accordance with the operation input to the master device, the information presentation system comprising:
a control means that controls tactile force transmission in the master device and the slave device;
a calculation means that calculates physical characteristics of a substance in contact with the slave device based on an external force input from an environment to the slave device while the slave device maintains a predetermined motion state; and
a presentation means that presents the physical characteristics of the substance calculated by the calculation means.

2. The information presentation system according to claim 1, wherein
the calculation means calculates elasticity as the physical characteristics of the substance when the slave device maintains a state of uniform motion as the predetermined motion state.

3. The information presentation system according to claim 1 or 2, wherein
the calculation means calculates viscosity as the physical characteristics of the substance when the slave device maintains a state of uniform acceleration motion as the predetermined action state.

4. The information presentation system according to any one of claims 1 to 3, wherein
the calculation means calculates inertia as the physical characteristics of the substance when the slave device maintains a state of uniform jerk motion as the predetermined action state.

5. The information presentation system according to any one of claims 1 to 4, wherein
the calculation means determines whether or not the slave device maintains the predetermined motion state based on a travel distance by which the slave device has moved after the predetermined motion state is started.

6. The information presentation system according to any one of claims 1 to 5, wherein
the presentation means presents the physical characteristics of the substance calculated by the calculation means and the physical characteristics of the substance as a reference in a comparable manner.

7. The information presentation system according to any one of claims 1 to 6, wherein
the presentation means presents the physical characteristics of the substance calculated by the calculation means to the operator of the master device.

8. An information presentation device comprising:
a control means that controls tactile force transmission in a master device to which an operation by an operator is input and a slave device that operates in accordance with the operation input to the master device;
a calculation means that calculates physical characteristics of a substance in contact with the slave device based on an external force input from an environment to the slave device while the slave device maintains a predetermined motion state; and
a presentation means that presents the physical characteristics of the substance calculated by the calculation means.

9. An information presentation method executed by an information presentation system including a master device to which an operation by an operator is input and a slave device that operates in accordance with the operation input to the master device, the information presentation method comprising:
a control step of controlling tactile force transmission in the master device and the slave device;
a calculation step of calculating physical characteristics of a substance in contact with the slave device based on an external force input from an environment to the slave device while the slave device maintains a predetermined motion state; and
a presentation step of presenting the physical characteristics of the substance calculated in the calculation step.

10. A program causing a computer to realize:
a control function that controls tactile force transmission in a master device to which an operation by an operator is input and a slave device that operates in accordance with the operation input to the master device;
a calculation function that calculates physical characteristics of a substance in contact with the slave device based on an external force input from an environment to the slave device while the slave device maintains a predetermined motion state; and
a presentation function that presents the physical characteristics of the substance calculated by the calculation function.
